(19) United States
(12) Patent Application Publication
(10) Pub. No.: US 2001/0002842 A1
(43) Pub. Date: Jun. 7, 2001

(54) Electronic endoscope system

(75) Inventor: Ryo Ozawa, Tokyo (JP)

Correspondence Address: GREENBLUM & BERNSTEIN, 1941 ROLAND CLARKE PLACE, RESTON, VA, 20191

(73) Assignee: ASAHI KOGAKU KOGYO KABUSHIKI KAISHA

(21) Appl. No.: 09/726,558

(22) Filed: Dec. 1, 2000

(30) Foreign Application Priority Data
Dec. 3, 1999 (JP) P11-344452

(51) Int. Cl. H04N 7/18; A61B 1/04

(52) U.S. Cl. 348/45; 600/109

(57) ABSTRACT

In an electronic endoscope system, a scope has a CCD image sensor provided at a distal end thereof to generate image-pixel signals, an image-signal processing unit produces a video signal based on the image-pixel signals, and a monitor displays an endoscope-image according to the video signal output from the processing unit. A scene-changing system changes a scene on the monitor between an endoscope-image-display scene and a patient-data-list-display scene. A storage system stores patient data forming a patient list which should be displayed on the monitor when the scene on the monitor is changed from the image-display scene to the list-display scene by the scene-changing system. A selection system selects individual patient data from the patient data list displayed on the monitor. A display-control system displays the selected individual patient data together with the endoscope-image on the monitor when the scene on the monitor is changed from the list-display scene to the image-display scene by the scene-changing system.

### BACKGROUND OF THE INVENTION

1. Field of the Invention

The present invention relates to an electronic endoscope system including an elongate flexible scope having a solid image sensor provided at a distal end thereof to generate image-pixel signals, an image-signal processing unit that produces a video signal on the basis of the image-pixel signals, and a TV monitor for reproducing and displaying an endoscope-image in accordance with the video signal.

2. Description of the Related Art

In such an electronic endoscope system, a CCD (charge-coupled-device) image sensor is usually utilized as the solid image sensor, and is associated with an objective lens system provided at the distal end of the flexible scope. Also, a flexible optical guide, formed of a bundle of optical fibers, is extended through the flexible scope, and is associated with a lighting lens system provided at the distal end of the flexible scope.

The image-signal processing unit includes a light source, such as a halogen lamp, a xenon lamp or the like, and when the flexible scope is connected to the image-signal processing unit, the proximal end of the optical light guide is optically connected to the light source. Thus, an object to be sensed by the CCD image sensor is illuminated by light radiating from the distal end of the optical light guide, and is focused as an optical image, on a light-receiving surface of the CCD image sensor, by the objective lens system.

The focused optical image is converted into a frame of analog image-pixel signals by the CCD image sensor, and the frame of analog image-pixel signals is read from the CCD image sensor at successively given regular time intervals. The successively-read frames of image-pixel signals are then fed to the image-signal processing unit, in which the frames of image-pixel signals are suitably processed to produce a video signal. The video signal is then fed from the image-signal processing unit to a TV monitor to reproduce an endoscope-image on the screen of the TV monitor.

As is well known, the CCD image sensor, used in the electronic endoscope system, is smaller than that of a CCD image sensor used in a usual TV camera. Namely, the number of image pixels included in one frame, obtained from the endoscope CCD image sensor, is less than the number of image pixels included in one frame, obtained from the TV camera CCD image sensor. Nevertheless, in the electronic endoscope system, a standard sized TV monitor normally is used, and thus an object sensed by the endoscope CCD image sensor is only reproduced and displayed on a partial area of the screen of the TV monitor.

Note, in reality, although a video signal is produced and prepared with respect to the overall area of the screen of the TV monitor, the video signal exhibits a pedestal or blanking level over the screen except for the partial area on which the endoscope-image is reproduced.

As is well known, the remaining area of the monitor screen except for the endoscope-image displaying area is usually utilized to display patient data, such as a name, ID number, age and so on. Displaying the patient data is significant because the endoscope-image is recorded on video tape or retrieved as a still image by an image-processing computer. In short, the patient data is necessary to accurately identify the source of the taped endoscope-image and the retrieved still image.

In order to display the patient data on the TV monitor, the image-signal processing unit includes a character generator in which character-pattern signals are generated on the basis of character code data corresponding to the patient data and added to the video signal, thereby displaying the patient data together with the endoscope image on the TV monitor.

Conventionally, the character code data, corresponding to the patient data, is manually input via a keyboard connected to the image-signal processing unit. The manual inputting of character code data is performed by a doctor, nurse or other operator, with reference to the patient's clinical record sheet, whenever a patient is medically examined using the electronic endoscope system. Of course, the amount of data input for each patient is relatively small, however, when many patients must be examined in a day, the consequence of the volume of patient data to be input cannot be overlooked as the efficiency of the medical examination is diminished due to the manual inputting of large amounts of patient data.

### SUMMARY OF THE INVENTION

Therefore, an object of the present invention is to provide an electronic endoscope system constituted such that patient data can be easily and quickly displayed, together with an endoscope-image, on a TV monitor at the beginning of a medical examination of each individual patient.

In accordance with the present invention, there is provided an electronic endoscope system including a scope having a solid image sensor provided at a distal end thereof to generate image-pixel signals, an image-signal processing unit that produces a video signal based on the image-pixel signals, and a monitor for reproducing and displaying an endoscope-image in accordance with the video signal output from the image-signal processing unit. The electronic endoscope system comprises a scene-changing system that changes a scene on the monitor between an endoscope-image-display scene and a patient-data-list-display scene, a storage system that stores patient data forming a patient data list which is displayed on the monitor when the scene on the monitor is changed from the endoscope-image-display scene to the patient-data-list-display scene by the scene-changing system, a selection system that selects individual patient data from the patient data list displayed on the monitor, and a display-control system that displays the selected individual patient data together with the endoscope-image on the monitor when the scene on the monitor is changed from the patient-data-list-display scene to the endoscope-image-display scene by the scene-changing system.

The electronic endoscope system may further comprise an editing system that edits the patient data, forming the patient data list, stored in the storage system.

Preferably, the production of the video signal is performed by the image-signal processing unit such that as much patient information as possible is included in the patient data list to be displayed on the TV monitor when the scene on the monitor is changed from the endoscope-image-display scene to the patient-data-list-display scene by the scene-changing system.

Optionally, the electronic endoscope system may further comprise a clock-pulse generator that produces first and second series of clock pulses, having different frequencies, such that the video signal is output from the image-signal processing unit to the monitor in accordance with either of the series of clock pulses, the first series of clock pulses having a higher frequency than that of the second series of clock pulses, a clock-pulse-selection system that selects either the first or second series of clock pulses to be output from the clock-pulse generator in accordance with a number of image-pixel signals obtained from the image sensor, and a clock-pulse-selection-controller that controls the clock-pulse-selection system such that the first series of clock pulses having the higher frequency is forcibly output from the clock-pulse generator whenever the scene on the monitor is changed from the endoscope-image-display scene to the patient-data-list-display scene by the scene-changing system.

Preferably, the selection system includes an indicator system that visually indicates a patient data to be selected from the patient data list, a manual operation system that controls the indication of the patient data to be selected from the patient data list, and a manual settlement system that manually settles the indication of the patient data to be selected from the patient data list.

The selection system may further include an editing system that edits the patient data, forming the patient list patient, stored in the storage system, and a determination system that determines whether editing of the patient data is performed by the editing system after an activation of the manual settlement system. The editing of the patient data is settled by an activation of the manual settlement system when the performance of the editing of the patient data is confirmed by the determination system.

### BRIEF DESCRIPTION OF THE DRAWINGS

The object and other objects of the present invention will be better understood from the following description, referring to the accompanying drawings, in which:

FIG. 1 is a schematic block diagram of an electronic endoscope system, according to the present invention, including an elongate flexible scope, an image-signal processing unit and a TV monitor;

FIG. 2 is a view showing, by way of an example, an endoscope-image-display on the TV monitor;

FIG. 3 is a view showing, by way of an example, a patient-data-list-display on the TV monitor;

FIG. 4 is a view showing, by way of an example, a patient-data-list-display on the TV monitor in accordance with a series of low frequency clock pulses;

FIG. 5 is a block diagram showing in detail a part of the block diagram of FIG. 1, which is especially related to the present invention;

FIG. 6 is a flowchart of an initialization routine in a system controller of the image-signal processing unit;

FIG. 7 is a flowchart of a clock-pulse-setting routine executed in the system controller of the image-signal processing unit;

FIG. 8 is a part of a flowchart of a key-operation-monitoring routine executed in the system controller of the image-signal processing unit;

FIG. 9 is the remaining part of the flowchart of the key-operation-monitoring routine;

FIG. 10 is a flowchart of a patient-data-editing routine executed in the system controller of the image-signal processing unit;

FIG. 11 is a view showing, by way of an example, the patient-data-list-display on the TV monitor, when being changed from the endoscope-image-display thereof; and

FIG. 12 is a flowchart of a patient-data-selecting routine executed in the system controller of the image-signal processing unit.

### DESCRIPTION OF THE PREFERRED EMBODIMENTS

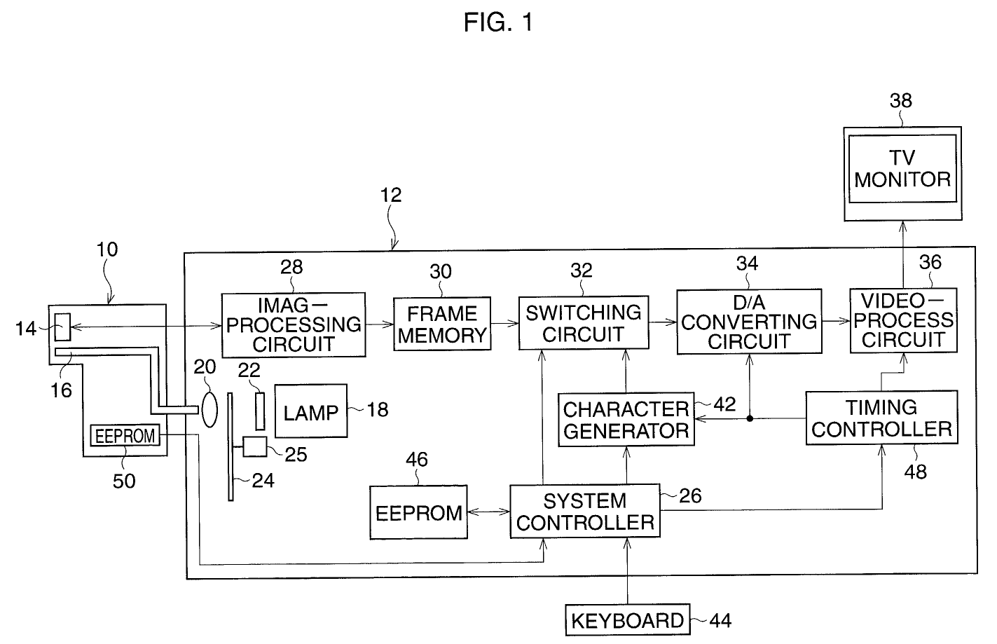

With reference to FIG. 1, an electronic endoscope system according to the present invention is schematically shown as a block diagram. The electronic endoscope system comprises an elongate scope 10 having a flexible conduit, and an image-signal processing unit 12 to which the scope 10 is detachably connected. The scope 10 is representative of various types of scope, used for bronchial, esophageal, gastro, colon, etc inspections, and these various types of scope use the image-signal processing unit 12 in common.

The scope 10 has a solid image sensor 14, such as a CCD (charge-coupled-device) image sensor, provided at a distal end of the flexible conduit thereof, and the CCD image sensor 14 is associated with an objective lens system (not shown). Also, the scope 10 includes a flexible optical light guide 16 extended therethrough and formed of a bundle of optical fibers. The optical light guide 16 terminates with a light-radiating end face at the distal end of the flexible conduit of the scope 10, and is associated with a lighting lens system (not shown) provided thereat. When the connection is established between the scope 10 and the image-signal processing unit 12, the proximal end of the optical light guide 16 is optically connected to a white-light lamp 18, such as a halogen lamp, xenon lamp or the like, provided in the image-signal processing unit 12, whereby light is emitted from the light-radiating end face of the optical light guide 16.

As shown in FIG. 1, a condenser lens 20 and a diaphragm 22 are provided between the lamp 18 and the proximal end of the optical light guide 16. The condenser lens 20 is used to converge the light, emitted from the lamp 18, on the proximal end of the optical light guide 16. The diaphragm 22 is used to adjust an amount of light directed from the lamp 18 to the proximal end of the optical light guide 16, i.e. the amount of the illuminating-light radiating from the distal end of the optical light guide 16 can be regulated by the diaphragm 22.

In this embodiment, since the CCD image sensor 14 is constituted as a monochromatic CCD image sensor, an RGB field sequential-type color imaging method is introduced in the electronic endoscope system, thereby obtaining a full color image from the monochromatic CCD image sensor 14. Thus, a rotary RGB color filter disk 24 is interposed between the condenser lens 20 and the diaphragm 22. The rotary RGB color filter disk 24 has three sector-shaped color filters, i.e. red, green and blue filters, and these color filters are circumferentially and uniformly arranged such that three centers of the color filters are spaced from each other at regular angular intervals of 120 degrees, with a sector between two adjacent color filters being constructed as a light-shielding area.

The rotary RGB color filter disk 24 is rotated by a suitable electric motor 25, such as a servo-motor, a stepping motor or the like, at a given rotational frequency in accordance with a used image-reproduction method, such as the NTSC system, the PAL system and so on, whereby an object to be sensed by the CCD image sensor 14 is sequentially illuminated with red, green and blue lights. Namely, red, green and blue optical images are sequentially and cyclically focused on the light-receiving surface of the CCD image sensor. Note, in the NTSC system, the rotational frequency of the color filter disk 24 is 30 Hz, and, in the PAL system, the rotational frequency of the color filter disk 24 is 25 Hz.

As shown in FIG. 1, the image-signal processing unit 12 is provided with a system controller 26, which may be constituted as a microcomputer, used to control the electronic endoscope system as a whole and comprising, for example, a central processing unit (CPU), a read-only memory (ROM) for storing programs and constants, a random-access memory (RAM) for storing temporary data, and an input/output interface circuit (I/O).

Also, the image-signal processing unit 12 is provided with an image-signal processor including an image-processing circuit 28, a frame memory 30 and a switching circuit 32, a digital-to-analog converting circuit 34 and a video-process circuit 36. As shown in FIG. 1, when the connection between the scope 10 and the image-signal unit 12 is established, the CCD image sensor 14 is connected to the image-processing circuit 28.

While the red, green and blue optical images are cyclically and sequentially focused on the light-receiving surface of the CCD image sensor 14, each of the red, green and blue optical images is converted into a frame of monochromatic (red, green, blue) analog image-pixel signals by the CCD image sensor 14, and the monochromatic analog image-pixel signals are read from the CCD image sensor 14 over consecutive light-shielding time periods corresponding to the light-shielding areas between two adjacent color filters of the color filter disk 24.

The read monochromatic analog image-pixel signals are fed to the image-processing circuit 28, in which the analog image-pixel signals are subjected to various image-processings, such as gamma-correction, white-balance correction, profile-enhancing processing and so on. Then, the processed monochromatic analog image-pixel signals are converted into monochromatic digital image-pixel signals by an analog-to-digital converter provided in the image-processing circuit 28.

The monochromatic digital image-pixel signals are stored in the frame memory 30. In this frame memory 30, three frame memory sections are defined for the storage of red, green and blue digital image-pixel signals, respectively. In short, the monochromatic digital image-pixel signals are stored in a corresponding frame memory section defined in the frame memory 30.

While the respective red, green and blue digital image-pixel signals are successively stored in the memory sections of the frame memory 30, the red, green and blue digital image-pixel signals are simultaneously read from the respective memory sections of the frame memory 30 as a red digital video signal component, a green digital video signal component and a blue digital video signal component, respectively, which form primary color video signal components of a component-type color video signal.

The red, green and blue digital video signal components, read from the frame memory 30, are input to the switching circuit 32, which is operated by the system controller 26. Usually, the switching circuit 32 is in an ON-state, and thus the red, green and blue digital video signal components are output from the switching circuit 32. When the switching circuit 32 is switched from the ON-state to an OFF-state by the system controller 26, the output of red, green and blue digital video signal components from the switching circuit 32 is stopped.

While the red, green and blue digital video signal components are output from the switching circuit 32, these digital video signal components are input to the digital-to-analog converting circuit 34, and are converted into red, green and blue analog video signal components, respectively.

The converted red, green and blue analog video signal components are input to the video-process circuit 36, in which the analog video signal components are suitably processed. Also, in the video-process circuit 36, a compound-synchronizing-signal component including various synchronizing signals, such as a horizontal synchronizing signal, a vertical synchronizing signal and so on, is combined with the processed red, green and blue analog video signal components, thereby producing a component-type color video signal. This component-type color video signal is fed from the video-process circuit 36 to a TV monitor 38, and a color endoscope-image is reproduced and displayed on a screen of the TV monitor 38 in accordance with the component-type color video signal.

As is well known, the CCD image sensor 14, used in the electronic endoscope system, is smaller than that of a CCD image sensor used in a usual TV camera. Namely, the number of image pixels included in one frame, obtained from the endoscope CCD image sensor, is less than a number of image pixels included in one frame, obtained from the TV camera CCD image sensor. Nevertheless, in the electronic endoscope system, the TV monitor 38 has a standard size, and thus the endoscope-image, sensed by the CCD image sensor 14, is only displayed on a partial area of the screen of the TV monitor 38.

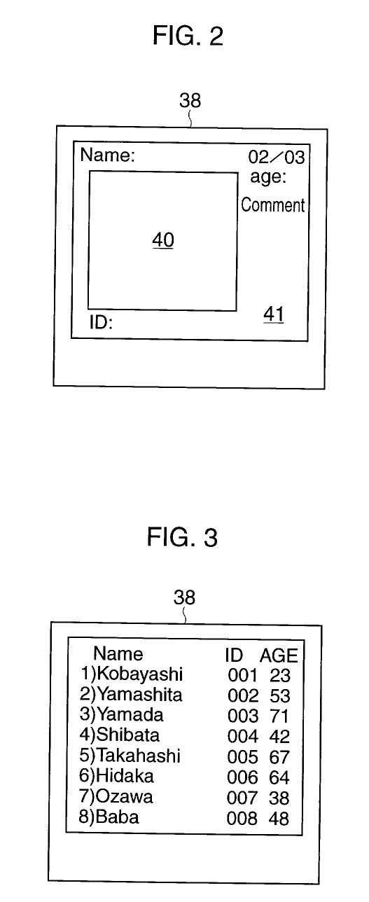

With reference to FIG. 2, a view of the screen of the TV monitor 38 is shown. In this drawing, the area of the screen, on which the endoscope-image is displayed, is generally indicated by reference 40. In reality, the component-type color video signal, output from the video-process circuit 36, is produced and prepared with respect to the overall area of the monitor screen, but the red, green and blue video signal components exhibit a pedestal or blanking level over the remaining area 41 of the screen except for the endoscope-image-displaying area 40.

As shown in FIG. 2, the remaining area 41 is used as a character-information-displaying area on which various character-information items, such as “Name:”, “age:”, “ID:”, “Comment” and “02/03”, are displayed. To this end, as shown in FIG. 1, the image-signal processing unit 12 is provided with a character generator 42, which is operated under control of the system controller 26 to generate digital color character-pattern video signal components corresponding to the character-information items. The generated digital color character-pattern video signal components are output from the character generator 42 to the switching circuit 32, and are added to the red, green and blue digital video signal components so that the character-information items, are displayed on the character-information-displaying area 41 of the TV monitor 38.

In the example shown in FIG. 2, each of the character-information items “Name:”, “age:”, “ID:” and “Comment” is dealt with as a fixed character-information item, and character code data, corresponding to each fixed character-information item, is stored in the ROM of the system controller 26.

On the other hand, variable character-information is displayed adjacent to each fixed character-information item on the character-information-displaying area 41 of the TV monitor 38. For example, a patient's name is displayed as a variable character-information item adjacent to the fixed character-information item “Name:”, a patient's age is displayed as a variable character-information item adjacent to the fixed character-information item A “age:”, and a patient's ID number is displayed as a variable character-information item adjacent to the fixed character-information-item “ID:”. Also, a medical examination comment is displayed as a variable character-information item adjacent to the fixed character-information item “Comment”, if necessary. Character code data, corresponding to each variable character-information item, is input to the system controller 26 through a keyboard 44.

Note, although the character-information item “02/03”, representing a date, is dealt with as a variable character-information item, character code data, corresponding to the variable character-information item “02/03”, is produced and renewed periodically by a built-in clock device of the system controller 26.

In this embodiment, character code data, corresponding to the patient's variable character-information items (i.e. names, ages and ID numbers) are input through the keyboard 44 prior to the examinations, and are stored as a patient data list or a patient data table in a non-volatile memory, such as an electrically erasable programmable read-only memory (EEPROM) 46.

As mentioned above, when the switching circuit 32 is switched from the ON-state to the OFF-state by the system controller 26, the output of red, green and blue digital video signal components from the switching circuit 32 is stopped. At this time, the character code data, forming the patient data list, are read from the EEPROM 46, and input to the character generator 42, in which digital color character-pattern video signal components are generated on the basis of the character code data forming the patient data list.

The generated digital color character-pattern video signal components are output from the character generator 42 to the switching circuit 32 and fed to the digital-to-analog converting circuit 34, in which the digital color character-pattern video signal components are converted into analog color character-pattern video signal components. Then, in the video-process circuit 36, the compound-synchronizing-signal component is added to the analog color character-pattern video signal components, thereby producing a component-type color character-pattern video signal, which is fed to the TV monitor 38.

Thus, as shown in FIG. 3, by way of example, a patient data list are displayed on the TV monitor 38 in a predetermined format in accordance with the component-type color character-pattern video signal. Note, as is apparent from FIG. 3, the patient data list is displayed over the whole of the monitor screen.

In short, while the switching circuit 32 is in the ON-state, the endoscope-image is displayed on the partial area 40 of the TV monitor 38, as shown in FIG. 2. However, as soon as the switching circuit 32 is switched from the ON-state to the OFF-state, the screen of the TV monitor 38 is changed to the patient-data-list-display as shown in FIG. 3.

As shown in FIG. 1, the image-signal processing unit 12 is provided with a timing controller 48, which is operated under control of the system controller 26. The timing controller 48, a clock-pulse generator, produces and outputs plural series of control clock pulses, having various frequencies, to sequentially and systematically operate the image-signal processor including the elements 28, 30, 32, 34 and 36.

For example, although the image-processing circuit 28 and the timing controller 48 are not shown as being connected to each other in FIG. 1, the reading of the image-pixel signals from the CCD image sensor 14 is performed in accordance with a series of clock pulses output from the timing controller 48, and the various image-processings are performed in the image-processing circuit 28 in accordance with a series of clock pulses output from the timing controller 48. Also, in the analog-to-digital converter included in the image-processing circuit 28, the conversion of the analog image-pixel signals to the digital image-pixel signals is performed in accordance with a series of clock pulses output from the timing controller 48.

Similarly, although the frame memory 30 and the timing controller 48 are not shown as being connected to each other in FIG. 1, the storage of the digital pixel signals in the frame memory 30 is performed in accordance with a series of clock pulses output from the timing controller 48, and the reading of the digital pixel signals from the frame memory 30 is performed in accordance with a series of clock pulses output from the timing controller 48.

Also, as shown in FIG. 1, the digital-to-analog converting circuit 34 and the character generator 42 are connected to the timing controller 48, the conversion of the digital color video signal components to the analog color video signal components in the digital-to-analog converting circuit 34 and the output of the digital color character-pattern signal components from the character generator 42 to the switching circuit 32 are performed in accordance with a series of clock pulses, output from the timing controller 48, with the series of clock pulses having a given frequency corresponding to the number of digital pixel-signals clock pulse included in a one-horizontal line of each digital (red, green, blue) video signal component.

Namely, the series of clock pulses, used for outputting the digital color character-pattern signal components from the character generator 42 to the switching circuit 32, must have the same frequency as that of the series of clock pulses used for the conversion of digital color video signal components to analog color video signal components in the digital-to-analog converting circuit 34, before the digital color character-pattern signal components can be added to the digital color video signal components with the proper timing.

Further, the timing controller 48 generates a compound-synchronizing signal component and outputs it to the video-process circuit 36, in which the compound-synchronizing signal component is combined with the analog component-type color video signal components output from the digital-to-analog converting circuit 34, resulting in the component-type color video signal, as already stated.

As mentioned previously, the scope 10 represents various types of scope, used for bronchial, esophageal, gastro, colon, etc examinations, and these various types of scope use the image-signal processing unit 12 in common. The various types of scope are sorted into two groups: one group features a relatively-large-sized CCD image sensor (14) ; and the other group features a relatively-small-sized CCD image sensor (14). Of course, the total number of image-pixel signals obtained from the relatively-large-sized CCD image sensor is more than the total of image-pixel signals obtained from the relatively-small-sized CCD image sensor.

When a scope 10 having the relatively-large-sized CCD image sensor (14), is connected to the image-signal processing unit 12, a first series of clock pulses having a first frequency according to the number of image-pixel signals obtained from the relatively-large-sized CCD image sensor, must be output from the timing controller 48 to the digital-to-analog converting circuit 34 and the character generator 42.

Similarly, when a scope 10 having the relatively-small-sized CCD image sensor (14), is connected to the image-signal processing unit 12, a second series of clock pulses having a second frequency according to the number of image-pixel signals obtained from the relatively-small-sized CCD image sensor, must be output from the timing controller 48 to the digital-to-analog converting circuit 34 and the character generator 42.

Of course, the first frequency is higher that the second frequency because the number of image-pixel signals to be processed by the first series of clock pulses is larger than the number of image-pixel signals to be processed by the second series of clock pulses. Thus, the timing controller 48 is constituted so as to selectively output one of the first series of clock pulses and the second series of clock pulses under control of the system controller 26 in accordance with the type of scope 10 connected to the image-signal processing unit 12.

As shown in FIG. 1, the scope 10 is provided with a non-volatile memory, such as an electrically erasable programmable read-only memory (EEPROM) 50, in which clock-pulse-frequency-indicating flag data is stored to identify whether the scope 10 has a relatively-large-sized CCD image sensor or a relatively-small-sized CCD image sensor. When the scope 10 is connected to the image-signal processing unit 12, the EEPROM 50 is connected to the system controller 26, whereby the clock-pulse-frequency-indicating flag data is retrieved from the EEPROM 50 by the system controller 26. Thus, it is possible for the system controller 26 to determine whether the first series of clock pulses or the second series of clock pulses should be output from the timing controller 48.

Note, the EEPROM 50 may store various data peculiar to the scope concerned, such as factors for the white-balance correction which is performed in the image-processing circuit 28.

In this embodiment, the patient data list is displayed on the TV monitor 28 in accordance with the first series of clock pulses having a higher frequency than that of the second series of clock pulses, because as much patient information as possible should be included in the patient data list to be displayed on the TV monitor 38. Accordingly, while displaying an endoscope-image on the TV monitor 38 in accordance with the second series of clock pulses, if the scene on the TV monitor 38 is changed from the endoscope-image-display to a patient-data-list-display (FIG. 3) by switching the switching circuit 32 from the ON-state to the OFF-state, the timing controller 48 switches output of clock pulses from the second series of clock pulses to the first series of clock pulses. Namely, display of the patient-data-list on the TV monitor 38 uses the first series of clock pulses as it has a higher frequency.

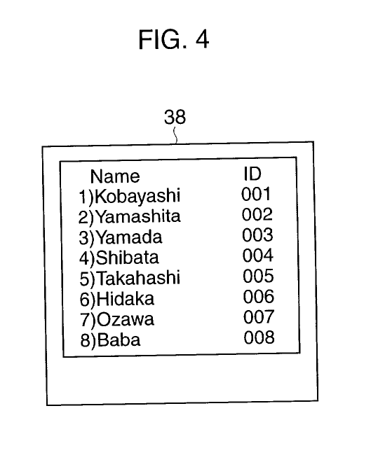

As shown in FIG. 4, by way of example, if the patient-data-list is displayed on the TV monitor 38 in accordance with the second series of clock pulses, having the lower frequency, a part of the patient data list cannot be displayed on the TV monitor 38. As an example, in FIG. 4, the patient's age data is omitted from the patient data list.

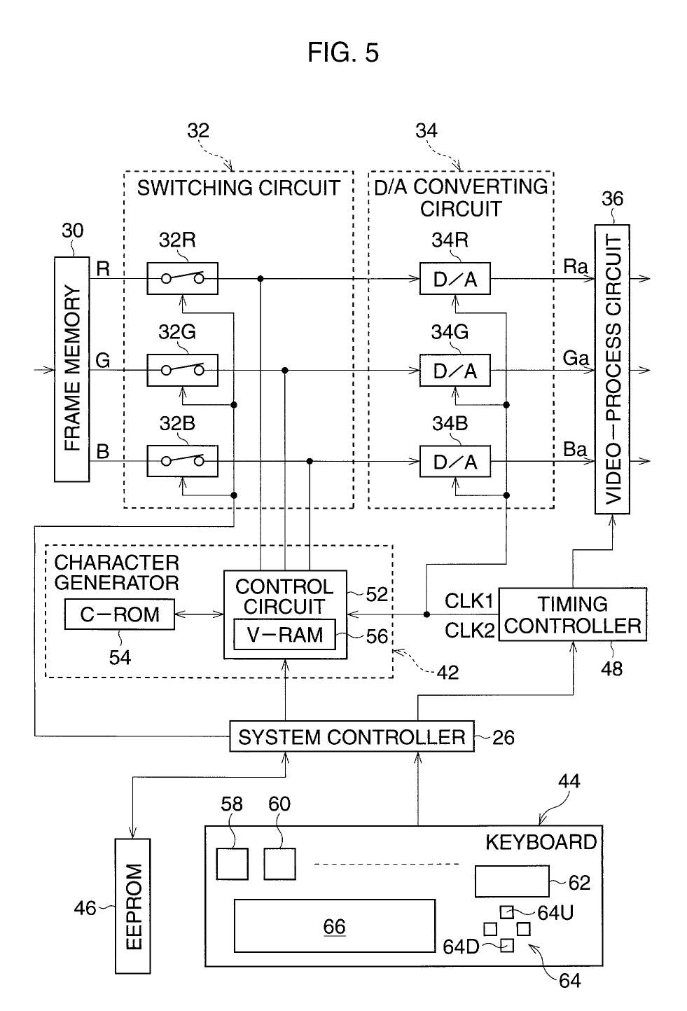

With reference to FIG. 5, a significant part of the block diagram of FIG. 1 is shown in detail.

The switching circuit 32 includes three ON/OFF switch elements 32R, 32G and 32B which are provided in output lines for the digital red, green and blue video signal components (R, G and B), extended from the frame memory 30. The ON or OFF state of each switch element (32R, 32G, 32B) is controlled by a scene-changing signal output from the system controller 26. In particular, when the scene-changing signal is low, all the ON/OFF switch elements 32R, 32G and 32B are turned ON, and thus output of the digital color video signal components from the switching circuit 32 to the digital-to-analog converting circuit 34 is allowed. When the scene-changing signal is changed from the low level to a high level, all the ON/OFF switch elements 32R, 32G and 32B are turned OFF, and thus the output of the digital color video signal components from the switching circuit 32 to the digital-to-analog converting circuit 34 is stopped.

The digital-to-analog converting circuit 34 includes three digital-to-analog (D/A) converters 34R, 34G and 34B, the input terminals of which are connected to output terminals of the ON/OFF switch elements 32R, 32G and 32B, respectively. While the ON/OFF switch elements 32R, 32G and 32B are in the ON-state, the respective digital color video signal components (R, G and B) are fed to the digital-to-analog converting circuit 34, and are converted into analog color video signal components (Ra, Ga and Ba) by the D/A converters 34R, 34G and 34B, with the converted signal components being fed to the video-process circuit 36. As stated above, in the video-process circuit 36, the compound-synchronizing signal component, output from the timing controller 48, is combined with the analog color video signal components (Ra, Ga and Ba), resulting in the production of the component-type color video signal.

The conversion of the digital color video signal components (R, G and B) into the analog monochromatic video signal components (Ra, Ga and Ba) by the D/A converters 32R, 32G and 32B is performed in accordance with either the first series of clock pulses or the second series of clock pulses, output from the timing controller 48, which are symbolically indicated and represented by references CLK1 and CLK2 in FIG. 5. Of course, as is apparent from the foregoing, when the analog monochromatic video signal components (Ra, Ga and Ba) are derived from a relatively-large-size CCD image sensor (14), the first series of clock pulses CLK1 is output from the timing controller 48, and, when the analog monochromatic video signal components (Ra, Ga and Ba) are derived from a relatively-small-size CCD image sensor (14), the second series of clock pulses CLK2 is output from the timing controller 48.

As shown in FIG. 5, the character generator 42 includes a control circuit 52 and a character ROM (C-ROM) 54. The control circuit 52 is formed as a microcomputer having a video RAM (V-RAM) 56. When character code data is written in an address of the V-RAM 56, the control circuit 52 outputs the character code data to the C-ROM 54, in which the character code data is converted into character-pattern signal data. Then, the control circuit 52 produces digital red, green and blue character-pattern video signal components from the character-pattern signal data, and outputs then to the output lines for the digital red, green and blue video signal components (R, G and B), extending from the ON/OFF switch elements 32R, 32G and 32B.

While the ON/OFF switch elements 32R, 32G and 32B are in the ON-state, character code data, corresponding to each of the fixed character-information item “Name:”, “age:”, “ID:” and “Comment”, is read from the ROM of the system control 26, and written in a predetermined address of the V-RAM 56, in which the character code data is converted into character-pattern signal data corresponding to each fixed character-information item (“Name:”, “age:”, “ID:”, “Comment”). Then, the control circuit 52 produces digital red, green and blue character-pattern video signal components from the character-pattern signal data, and outputs them to the output lines extending from the ON/OFF switch elements 32R, 32G and 32B, whereby the digital red, green and blue character-pattern video signal components are added to the digital red, green and blue video signal components (R, G and B). Thus, as shown in FIG. 2, by way of example, the endoscope-image is displayed together with the fixed character-information items “Name:”, “age:”, “ID:” and “Comment” on the TV monitor 38.

Note, of course, output of the digital red, green and blue character-pattern video signal components from the character generator 42 is performed in accordance with either the first series of clock pulses CLK1 or the second series of clock pulses CLK2. Also, note, the address in the v-RAM 56, to which each character code data is written, corresponds to a location on the TV monitor 38, at which a fixed character-information item (“Name:”, “age:”, “ID:”, “Comment”), corresponding to each character code data, is displayed.

On the other hand, when the ON/OFF switch elements 32R, 32G and 32B are turned OFF, i.e. when the scene-changing signal is changed from the low level to the high level, the output of the digital color video signal components (R, G and B) from the ON/OFF switch elements 32R, 32G and 32B is stopped. The V-RAM 56 is immediately cleared. Then, all the character code data forming the patient data list are read from the EEPROM 46, and are written in predetermined addresses of the V-RAM 56. Thus, the screen on the TV monitor 38 is changed from the endoscope-image-display scene to the patient-data-list-display scene as shown in FIG. 3. Note, at all times, the patient-data-list display scene is displayed on the TV monitor 38 in accordance with the first series of clock pulses CLK1, as stated above.

In this embodiment, changing the endoscope-image-display scene (FIG. 2) to the patient-data-list-display scene (FIG. 3) and returning the patient-data-list-display scene to the endoscope-image-display scene 38 are performed by depressing either two function keys 58 or 60 on the keyboard 44. For example, when the function key 58 is depressed, the scene-changing signal is changed from the low level to the high level, and, when the function key 58 is depressed again, the scene-changing signal is returned from the high level to the low level. The same is true for the key 60.

The keyboard 44 is provided with an “Enter” key 62 and four shift keys, generally indicated by reference 64. Two of the four shift keys 64, i.e. an up-shift key and a down-shift key, which especially relate to the present invention, are indicated by reference 64U and 64D. Also, the keyboard 44 is provided with a character-inputting key area, generally indicated by reference 66, in which character-inputting keys and other keys are arranged.

The function key 58, the “Enter” key 62, the up-shift key 64U, the down-shift key 64D and various character-inputting keys (66) are used when editing the patient data list, and the function key 60, the “Enter” key 62, the up-shift key 64U and the down-shift key 64D are used when selecting patient data from the patient data list, as stated in detail hereinafter.

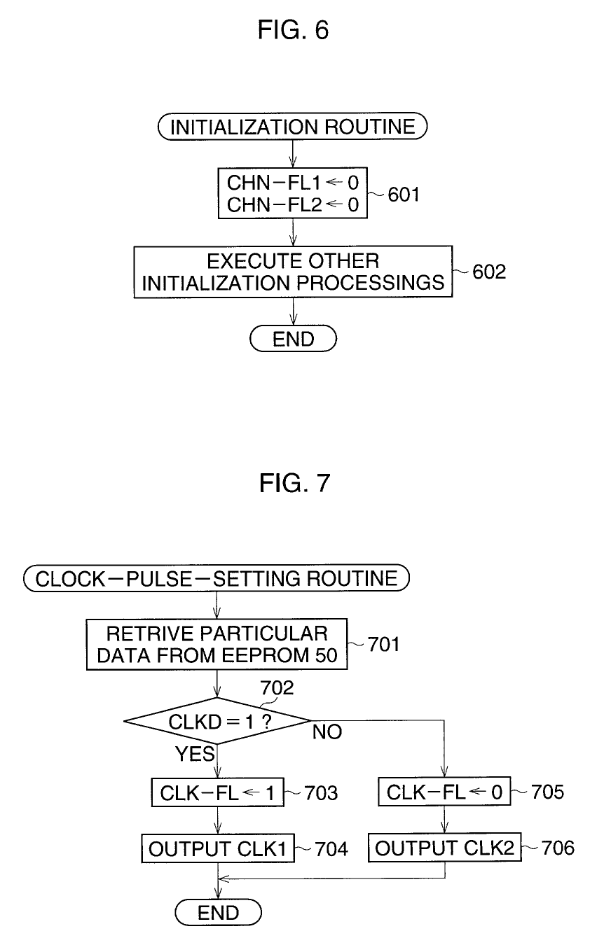

FIG. 6 shows a flowchart of an initialization routine, which is only executed once by the system controller 26 when the image-signal processing unit 12 is initially powered ON.

At step 601, a first scene-changing flag CHN-FL1 and a second scene-changing flag CHN-FL2 are initialized to “0”. Then, at step 602, other initialization processes, necessary to control the electronic endoscope system as a whole, are executed. After the execution of the other initialization processes, the routine ends.

Each of the first and second scene-changing flags CHN-FL1 and CHN-FL2 is used to indicate whether the scene-changing signal, output from the system controller 26 to the ON/OFF switch elements 32R, 32G and 32B, is in the low level or in the high level. When both the flags CHN-FL1 and CHN-FL2 are “0”, the scene-changing signal is in the low-level, i.e. the ON/OFF switch elements 32R, 32G and 32B are in the ON-state, so that the endoscope-image is displayed on the TV monitor 38 (FIG. 2). When one of the flags CHN-FL1 and CHN-FL2 is “1”, the scene-changing signal is in the high-level, i.e. the ON/OFF switch elements 32R, 32G and 32B are in the OFF-state, so that the patient data list is displayed on the TV monitor 38 (FIG. 3).

Note, as stated hereinafter, the respective first and second scene-changing flags CHN-FL1 and CHN-FL2 are associated with the function keys 58 and 60 on the key board 44.

FIG. 7 shows a flowchart of a clock-pulse-setting routine which is executed in the system controller 26 when it is detected that the scope 10 is connected to the image-signal processing unit 12. When the image-signal processing unit 12 is powered ON, with the scope 10 already connected to the image-processing unit 12, the clock-pulse-setting routine is executed consecutive to the initialization routine of FIG. 6. Also, when the scope 10 is connected to the image-signal processing unit 12, after the image-signal processing unit 12 is powered ON, the clock-pulse-setting routine is executed as soon as the connection is detected.

Note, the detection of the connection of the scope 10 to the image-signal processing unit 12 may be carried out by a suitable switch incorporated in a connector of the image-signal processing unit 12 to which the scope 10 is connected.

At step 701, data relevant to the connected scope 10 is retrieved from the EEPROM 50. The retrieved data includes a clock-pulse-frequency-indicating flag data CLKD to identify whether the image sensor 14 of the scope 10 is a relatively-large-sized CCD image sensor or a relatively-small-sized CCD image sensor. In this embodiment, when the scope 10 has the relatively-large-sized CCD image sensor, a setting of “1” is given to the flag data CLKD, and, when the scope 10 has the relatively-small-sided CCD image sensor, a setting of “0” is given to the flag data CLKD.

At step 702, it is determined whether the flag data CLKD is “1” or “0”. If CLKD=1, the control proceeds to step 703, in which a clock-pulse-selecting flag CLK-FL is set to “1”. Then, at step 704, the first clock pulses CLK1 is output from the timing controller 48.

At step 702, if CLKD=0, the control proceeds from step 702 to step 705, in which a clock-pulse-selecting flag CLK-FL is set to “0”. Then, at step 706, the second clock pulses CLK2 is output from the timing controller 48.

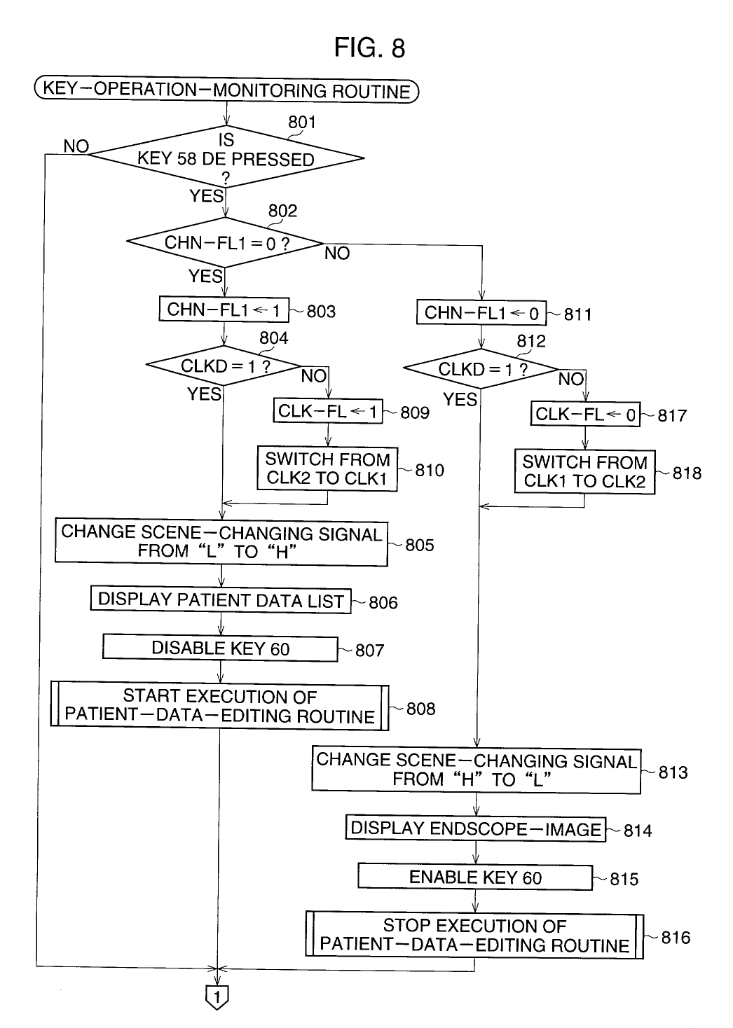

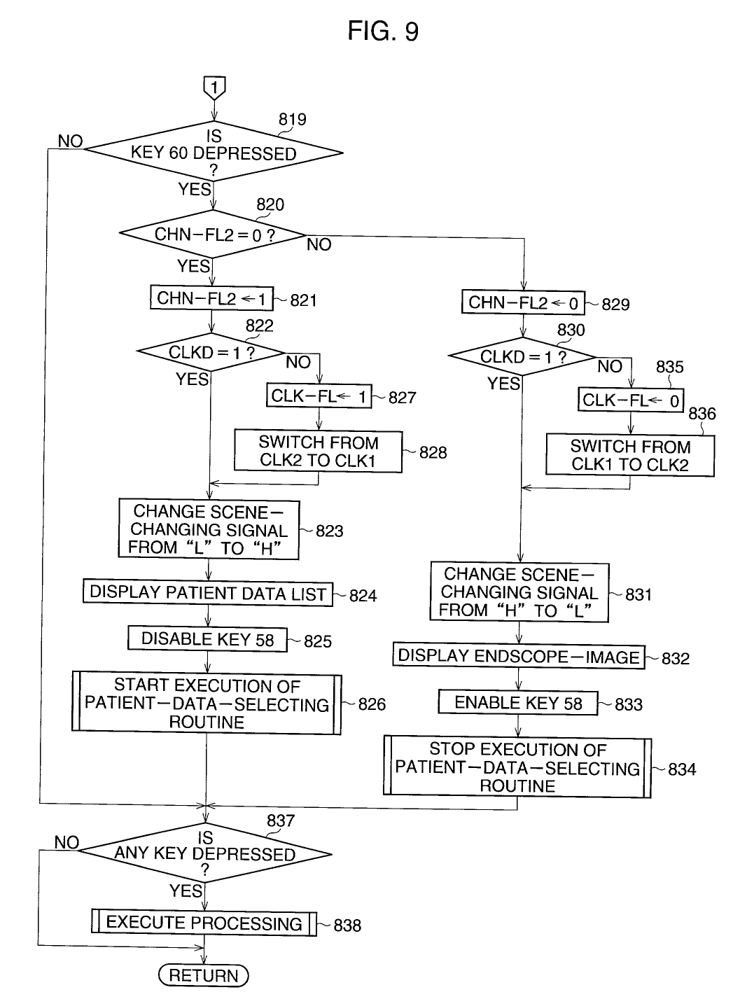

FIGS. 8 and 9 show a flowchart of a key-operation-monitoring routine which is formed as a time-interruption routine executed in the system controller 26 at regular suitable intervals, for example, 10 ms. Note that the execution of the key-operation-monitoring routine is consecutive to the initialization routine of FIG. 6, and is repeated every 10 ms as long as the image-signal processing unit 12 is powered ON.

At step 801, it is monitored whether the function key 58 has been depressed. When the depression of the function key 58 is not confirmed, the control skips to step 819 (FIG. 9), in which it is monitored whether the function key 60 has been depressed. When the depression of the function key 60 is not confirmed, the control skips to step 837, in which it is monitored whether another function key is depressed on the keyboard 44. When it is confirmed that any key is not depressed, the routine ends. Thereafter, although the execution of the routine is repeated every 10 ms, there is no progress until a function key is depressed on the keyboard 44.

At step 801, when the depression of the function key 58 is confirmed, the control proceeds to step 802, in which it is determined whether the first scene-changing flag CHN-FL1 is “0” or “1”. If CHN-FL1=0, i.e. if an endoscope-image is displayed on the TV monitor 38 (FIG. 2), the control proceeds to step 803, in which the first scene-changing flag CHN-FL1 is set to “1”.

At step 804, it is determined whether the clock-pulse-frequency-indicating flag data CLKD is “1” or “0”. If CLKD=1, i.e. if the displaying of the endoscope-image on the TV monitor 38 is performed in accordance with the first clock pulses CLK1 having the high frequency, the control proceeds to step 805, in which the scene-changing signal, output from the system controller 26 to the ON/OFF switch elements 32R, 32G and 32B, is changed from the low level “L” to the high level “H”, thereby turning OFF the ON/OFF switch elements 32R, 32G and 32B. Namely, output of the digital color video signal components (R, G and B) from the ON/OFF switch elements 32R, 32G and 32B to the D/A converters 34R, 34G and 34B is discontinued.

At step 806, the scene on the TV monitor 38 is changed from the endoscope-image-display scene (FIG. 2) to a patient-data-list-display scene (FIG. 3). Namely, all the character code data, forming the patient data list, are read from the EEPROM 46, and are written at predetermined addresses in the V-RAM 56, whereby the patient data list is displayed on the TV monitor 38 (FIG. 3).

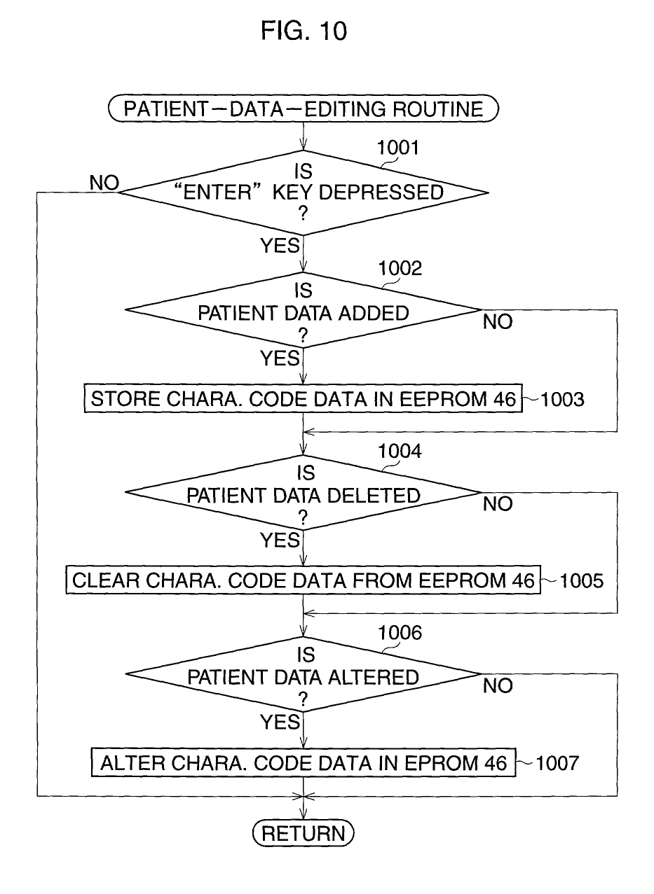

At step 807, the function key 60 is disabled, and then at step 808, execution of a patient-data-editing routine is started. Namely, whenever the first scene-changing flag CHN-FL1 is set to “1” by the depression of the function key 58 (step 803), i.e. whenever the scene on the TV monitor 38 is changed from the endoscope-image-display scene to the patient-data-list-display scene by depressing the function key 58, the function key 60 is disabled, and execution of the patient-data-editing routine is started. Note, the patient-data-editing routine is explained in detail with reference to FIG. 10 hereinafter.

At step 804, if the clock-pulse-frequency-indicating flag data CLKD is “0”, i.e. if the endoscope-image display on the TV monitor 38 is performed in accordance with the second clock pulses CLK2 having the lower frequency, the control proceeds from step 804 to step 809, in which the clock-pulse-selecting flag CLK-FL is set to “1”. Then, at step 810, the timing controller 48 switches output of clock pulses from the second clock pulses CLK2 to the first clock pulses CLK1. Thereafter, the control proceeds to step 805. Namely, display of the patient data list on the TV monitor 38 is always performed in accordance with the first clock pulses CLK1 having the higher frequency, as already stated.

At step 802, if CHN-FL1=0, i.e. if the patient data list is displayed on the TV monitor 38 (FIG. 3), the control proceeds from step 802 to step 811, in which the first scene-changing flag CHN-FL1 is set to “1”.

At step 812, it is determined whether the clock-pulse-frequency-indicating flag data CLKD is “1” or “0”. If CLKD=1, i.e. if the endoscope-image should be displayed on the TV monitor 38 in accordance with the first clock pulses CLK1 having the higher frequency, the control proceeds to step 813, in which the scene-changing signal, output from the system controller 26 to the ON/OFF switch elements 32R, 32G and 32B, is changed from the high level “H” to the low level “L”, thereby turning ON the ON/OFF switch elements 32R, 32G and 32B. Thus, output of the digital color video signal components (R, G and B) from the ON/OFF switch elements 32R, 32G and 32B to the D/A converters 34R, 34G and 34B is resumed, whereby the scene on the TV monitor 38 is returned from the patient-data-list-display scene (FIG. 3) to the endoscope-image-display scene (step 814).

When the scene on the TV monitor 38 is switched from the patient-data-list-display scene to the endoscope-image-display scene, all the character code data, forming the patient data list, are cleared from the V-RAM 56. Then, the character code data, corresponding to the fixed character-information items “Name:”, “age:”, “ID:” and “Comment”, are read from the ROM, and are written at predetermined addresses in the V-RAM 56, so that the fixed character-information items “Name:”, “age:”, “ID:” and “Comment” are displayed on the character-information-displaying area 41 of the TV monitor 38 (FIG. 2).

Note, of course, when the scene on the TV monitor 38 is changed from the endoscope-image-display scene to the patient-data-list-display scene (step 806), the character code data, corresponding to the fixed character-information items “Name:”, “age:”, “ID:” and “Comment”, are cleared from the V-RAM 56.

At step 815, the function key 60 is enabled, and then at step 816, execution of the patient-data-editing routine (FIG. 10) is stopped. Namely, whenever the first scene-changing flag CHN-FL1 is set to “0” by depressing the function key 58, i.e. whenever the scene on the TV monitor 38 is switched from the patient-data-list-display scene to the endoscope-image-display scene by depressing the function key 58, the function key 60 is enabled, and execution of the patient-data-editing routine (FIG. 10) is stopped.

At step 812, if the clock-pulse-frequency-indicating flag data CLKD is “0”, i.e. if the endoscope-image should be displayed on the TV monitor 38 in accordance with the second clock pulses CLK2 having the lower frequency, the control proceeds from step 812 to step 817, in which the clock-pulse-selecting flag CLK-FL is set to “0”. Then, at step 818, the timing controller 48 switches output of clock pulses from the first clock pulses CLK1 to the second clock pulses CLK2. Thereafter, the control proceeds to step 813. Namely, when the scene on the TV monitor 38 is switched from the patient-data-list-display scene (FIG. 3) to the endoscope-image-display scene (FIG. 2), the endoscope-image is displayed on the TV monitor 38 in accordance with the second clock pulses CLK2 having the lower frequency, as the clock-pulse-frequency-indicating flag data CLKD is “0”.

In short, whenever the function key 58 is depressed, the endoscope-image-display scene and the patient-data-list-display scene alternate on the screen of the TV monitor 38, and the patient-data-editing routine (FIG. 10) is executed only while the patient data list is displayed on the TV monitor 38.

At step 819, operation of the function key 60 is monitored. When depression of the function key 60 is confirmed, the control proceeds to step 820, in which it is determined whether the second scene-changing flag CHN-FL2 is “0” or “1”. If CHN-FL2=0, i.e. if an endoscope-image is displayed on the TV monitor 38 (FIG. 2), the control proceeds to step 821, in which the second scene-changing flag CHN-FL2 is set to “1”.

At step 822, it is determined whether the clock-pulse-frequency-indicating flag data CLKD is “1” or “0”. If CLKD=1, i.e. if the endoscope-image is displayed on the TV monitor 38 in accordance with the first clock pulses CLK1 having the higher frequency, the control proceeds to step 823, in which the scene-changing signal, output from the system controller 26 to the ON/OFF switch elements 32R, 32G and 32B, is changed from the low level “L” to the high level “H”, thereby turning OFF the ON/OFF switch elements 32R, 32G and 32B. Namely, output of the digital color video signal components (R, G and B) from the ON/OFF switch elements 32R, 32G and 32B to the D/A converters 34R, 34G and 34B is discontinued.

At step 824, the scene on the TV monitor 38 is changed from the endoscope-image-display scene (FIG. 2) to a patient-data-list-display scene (FIG. 3). Namely, all the character code data, forming the patient data list, are read from the EEPROM 46, and are written at predetermined addresses in the V-RAM 56, whereby the patient data list is displayed on the TV monitor 38 (FIG. 3).

At step 825, the function key 58 is disabled, and then at step 808, execution of a patient-data-selecting routine is started. Namely, whenever the second scene-changing flag CHN-FL2 is set to “1” by depressing the function key 60 (step 821), i.e. whenever the scene on the TV monitor 38 is changed from the endoscope-image-display scene to the patient-data-list-display scene by depressing the function key 60, the function key 58 is disabled, and execution of the patient-data-selecting routine is started. Note, the patient-data-selecting routine is explained in detail with reference to FIG. 12 hereinafter.

At step 822, if the clock-pulse-frequency-indicating flag data CLKD is “0”, i.e. if the endoscope-image is displayed on the TV monitor 38 in accordance with the second clock pulses CLK2 having the lower frequency, the control proceeds from step 822 to step 827, in which the clock-pulse-selecting flag CLK-FL is set to “1”. Then, at step 828, the timing controller 48 switches output of clock pulses from the second clock pulses CLK2 to the first clock pulses CLK1. Thereafter, the control proceeds to step 823. Namely, the patient data list is always displayed on the TV monitor 38 in accordance with the first clock pulses CLK1 having the high frequency, as already stated.

At step 820, if CHN-FL2=0, i.e. if the patient data list is displayed on the TV monitor 38 (FIG. 3), the control proceeds from step 820 to step 829, in which the second scene-changing flag CHN-FL2 is set to “0”.

At step 830, it is determined whether the clock-pulse-frequency-indicating flag data CLKD is “1” or “0”. If CLKD=1, the control proceeds to step 831, in which the scene-changing signal, output from the system controller 26 to the ON/OFF switch elements 32R, 32G and 32B, is changed from the high level “H” to the low level “L”, thereby turning ON the ON/OFF switch elements 32R, 32G and 32B. Thus, output of the digital color video signal components (R, G and B) from the ON/OFF switch elements 32R, 32G and 32B to the D/A converters 34R, 34G and 34B is resumed, whereby the scene on the TV monitor 38 is switched from the patient-data-list-display scene (FIG. 3) to the endoscope-image-display scene (step 832).

Similar to the aforementioned case, when the scene on the TV monitor 38 is switched from the patient-data-list-display scene to the endoscope-image-display scene, all the character code data, forming the patient data list, are cleared from the V-RAM 56. Then, the character code data, corresponding to the fixed character-information items “Name:”, “age:”, “ID:” and “Comment”, are read from the ROM, and are written at predetermined addresses in the V-RAM 56, so that the fixed character-information items “Name:”, “age:”, “ID:” and “Comment” are displayed on the character-information-displaying area 41 (FIG. 2). Also, of course, when the scene on the TV monitor 38 is changed from the endoscope-image-display scene to the patient-data-list-display scene (step 824), the character code data, corresponding to the fixed character-information items “Name:”, “age:”, “ID:” and “Comment”, are cleared from the V-RAM 56.

At step 833, the function key 58 is enabled, and then at step 816, execution of the patient-data-selecting routine (FIG. 12) is stopped. Namely, whenever the second scene-changing flag CHN-FL2 is set to “0” by depressing the function key 60, i.e. whenever the scene on the TV monitor 38 is switched from the patient-data-list-display scene to the endoscope-image-display scene endoscope-image by the depression of the function key 60, the function key 58 is enabled, and the execution of the patient-data-selecting routine (FIG. 12) is stopped.

At step 830, if the clock-pulse-frequency-indicating flag data CLKD is “0”, i.e. if the endoscope-image should be displayed on the TV monitor 38 in accordance with the second clock pulses CLK2 having the lower frequency, the control proceeds from step 830 to step 835, in which the clock-pulse-selecting flag CLK-FL is set to “0”. Then, at step 836, the timing controller 48 switches output of clock pulses from the first clock pulses CLK1 to the second clock pulses CLK2. Thereafter, the control proceeds to step 831. Namely, when the scene on the TV monitor 38 is switched from the patient-data-list-display scene (FIG. 3) to the endoscope-image-display scene (FIG. 2), the endoscope-image is displayed on the TV monitor 38 in accordance with the second clock pulses CLK2 having the lower frequency, as the clock-pulse-frequency-indicating flag data CLKD is “0”.

In short, whenever the function key 60 is depressed, the endoscope-image-display scene and the patient-data-list-display scene alternate on the screen of the TV monitor 38, and the patient-data-selecting routine (FIG. 12) is executed only while the patient data list is displayed on the TV monitor 38.

At step 837, when the depression of any key (except for the functions keys 58 and 60) is confirmed, the control proceeds to step 838, in which a process relevant to the depressed key is executed.

FIG. 10 shows a flowchart of the patient-data-editing routine, which is formed as a time-interruption routine executed in the system controller 26 at regular suitable intervals, for example, 20 ms. As stated above with reference to FIGS. 8 and 9, execution of the patient-data-editing routine is started when the scene on the TV monitor 38 is changed from the endoscope-image-display scene (FIG. 2) to the patient-data-list-display scene (FIG. 3) by depressing of the function key 58 (step 808), and is ended when the scene on the TV monitor 38 is switched from the patient-data-list-display scene (FIG. 3) to the endoscope-image-display scene (FIG. 2) by the next depression of the function key 58 (step 816).

By executing of the patient-data-editing routine, the patient data list may be edited. In this embodiment, editing includes addition, deletion and alteration of patient data in the patient data list, and is performed while watching the patient-data-list-display scene on the TV monitor 38.

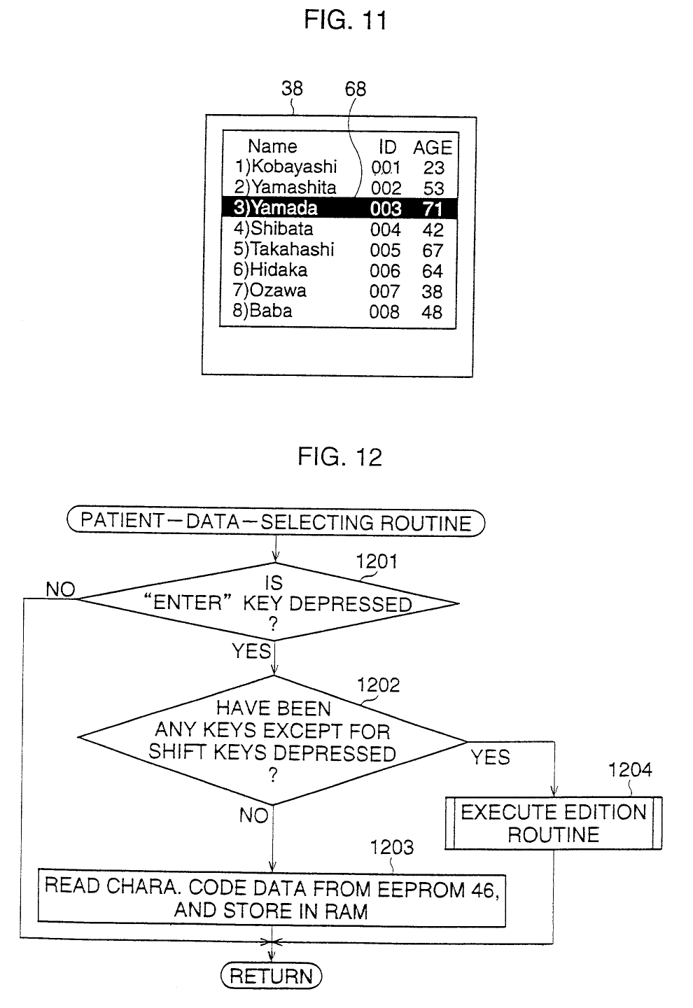

When the scene on the TV monitor 38 is changed from the endoscope-image-display scene to the patient-data-list-display scene, a reverse-display area 68 appears on the patient-data-list-display scene on the TV monitor 38, as shown in FIG. 11, by way of example. In this example, data of a patient “Yamada” is reversely displayed. The reverse-display area 68 can be moved by depressing the up-shift key 64U or the down-shift key 64D. When the up-shift key 64U is depressed, the reverse-display area 68 is moved up. Also, when the down-shift key 64D is depressed, the reverse-display area 68 is moved down. In short, by shifting the reverse-display area 68, data of a patient is selected from the displayed patient data list as the subject to be edited.

For example, when new patient data is added between a patient “Yamashita” and a patient “Yamada”, data of the patient “Yamada” is reversely displayed as shown in FIG. 11. Then, the data of new patient is input by operating keys on the character-inputting key area 66 such that the new patient's name, ID number and age are displayed between the patient “Yamashita” and the patient “Yamada”. Thereafter, by depressing the “Enter” key 62, character code data, corresponding to the new patient's name, ID number and age, are stored at given addresses in the EEPROM 46.

Also, for example, in order to delete the data of the patient “Yamada” from the patient data list, first, the data of patient “Yamada” is reversely displayed (FIG. 11). Then, when the “Enter” key 62 is depressed after depressing a key (not shown), allotted for deletion on the character-inputting key area 66, the deletion of the data for “Yamada” is carried out. Namely, the character code data, corresponding to the data for the patient “Yamada”, are cleared from the EEPROM 46.

Further, for example, in order to alter the data of the patient “Yamada” in the patient data list, first, the data for “Yamada” is reversely displayed (FIG. 11). Then, a patient's name, ID number and age are input by using keys on the character-inputting key area 66, so that the input data overwrites the displayed data. Thereafter, by depressing the “Enter” key 62, the character code data, corresponding to the data of the patient “Yamada” is replaced with character code data corresponding to the input data.

The patient-data-editing routine of FIG. 10 is now explained as follows:

At step 1001, it is monitored every 20 ms whether the “Enter” key 62 has been depressed. When the depression of the “Enter” key 62 is confirmed, the control proceeds to step 1002, in which it is determined whether new patient data has been added to the patient data list before the depression of the “Enter” key 62. When the addition of new patient data is confirmed, the control proceeds to step 1003, in which character code data, corresponding to the new patient's name, ID number and age, are stored at given addresses in the EEPROM 46.

When the addition of new patient data is not confirmed, the control skips step 1002 to step 1004, in which it is determined whether patient data has been deleted from the patient data list before the depression of the “Enter” key 62. When the deletion of patient data is confirmed, the control proceeds to step 1005, in which character code data, corresponding to the deleted patient data, is cleared from the EEPROM 46.

When the deletion of patient data is not confirmed, the control skips step 1004 to step 1006, in which it is determined whether patient data has been altered in the patient data list before the depression of the “Enter” key 62. When the alteration of patient data is confirmed, the control proceeds to step 1007, in which character code data, corresponding to the altered patient data, entered in the EEPROM 46.

When data alteration is not confirmed, the routine ends. Namely, when the “Enter” key 62 is depressed without any prior editing, the depression of the “Enter” key 62 is ignored.

The above-mentioned editing can be performed prior to medical examinations of patients. When a patient undergoes a medical examination using the electronic endoscope system, the data (name, ID number and age) to be displayed on the display area 41 of the TV monitor 38 is selected from the patient data list according to the patient-data-selecting routine of FIG. 12. In general, execution of the patient-data-editing routine is carried out prior to the medical examinations of patients, whereas execution of the patient-data-selecting routine is carried out prior to the medical examination of each patient. Nevertheless, in the patient-data-selecting routine, editing of the patient data list may be performed, for example, when an error is detected in the list, or when a new patient is urgently added to the list.

As stated hereinbefore with reference to FIGS. 8 and 9, the execution of the patient-data-selecting routine is started when the scene on the TV monitor 38 is changed from the endoscope-image-display scene to the patient-data-list-display scene by depressing the function key 60 (step 826), and is ended when the scene on the TV monitor 38 is switched from the patient-data-list-display scene to the endoscope-image-display scene by the next depression of the function key 60 (step 834). The patient-data-selecting routine also is formed as a time-interruption routine executed in the system controller 26 at regular suitable intervals, for example, 20 ms.

Similar to the patient-data editing routine of FIG. 10, when the scene on the TV monitor 38 is changed from the endoscope-image-display scene to the patient-data-list-display scene, a reverse-display area 68 appears on the patient-data-list-display scene on the TV monitor 38 (FIG. 11), and the reverse-display area 68 can be moved by depressing the up-shift key 64U or the down-shift key 64D. In short, by shifting the reverse-display area 68, a patient data is selected from the displayed patient data list.

The patient-data-selecting routine of FIG. 12 is now explained as follows:

At step 1201, it is monitored every 20 ms whether the “Enter” key 62 has been depressed every 20 ms. When the depression of the “Enter” key 62 is confirmed, the control proceeds step 1202, in which it is determined whether any keys except for the shift keys (especially 64U and 64D) have been depressed on the character-inputting key area 66 prior to the depression of the “Enter” key, i.e. whether character-inputting keys have been operated on the character-inputting key area 66 for editing of the patient data list prior to the depression of the “Enter” key.

When the depression of the character-inputting keys is not confirmed, i.e. when the editing of the patient data is not confirmed, the control proceeds to step 1203, in which character code data, corresponding to data of a patient selected by the reverse-display area 68, is read from the EEPROM 46, and are temporarily stored in the RAM of the system controller 26.

When the scene on the TV monitor 38 is switched from the patient-data-list-display scene to the endoscope-image-display scene by depressing the function key 60, i.e. when the patient-data-selecting routine is ended (step 834), the character code data are read from the RAM of the system controller 26, and are written at predetermined addresses in the V-RAM 56 of the character generator 42, whereby the patient data is displayed on the TV monitor 38. For example, when the data for patient “Yamada” is selected as shown in FIG. 11, the patient's name “Yamada” is displayed adjacent to the fixed character-information item “Name:”, the patient's ID number “003” is displayed adjacent to the fixed character-information item “ID:”, and the patient's age is displayed adjacent to the fixed character-information item “age:”.

At step 1202, when the editing of the patient data is confirmed before the depression of the “Enter” key 62 (step 1201), the control proceeds from step 1202 to step 1204, in which an edition routine, comprising steps 1002 to 1007 shown in FIG. 10, is executed.

As is apparent from the foregoing, according to the present invention, the patient data list can be previously prepared and stored in the EEPROM 46, and individual patient data can be displayed, together with an endoscope-image, on the TV monitor 38 by selecting the individual patient data from the patient data list. Namely, when a patient undergoes a medical examination using the electronic endoscope system, the data (name, ID number and age) can be easily and quickly displayed on the TV monitor 38 without any manual inputting of the data using character-inputting keys on the keyboard 44.

As is apparent from the foregoing, in the above-mentioned embodiment, data for each patient includes a name, an age and an ID number, and these variable character-information items are handled in a lump as the patient data when they are selected from the patient data list and displayed on the TV monitor. Of course, another piece of information for a patient may be included in the patient data as a variable character-information item.

Finally, it will be understood by those skilled in the art that the foregoing description is of preferred embodiments of the system, and that various changes and modifications may be made to the present invention without departing from the spirit and scope thereof.

The present disclosure relates to subject matters contained in Japanese Patent Applications No. 11-344452 (filed on Dec. 3, 1999) which is expressly incorporated herein, by reference, in its entirety.

1. An electronic endoscope system including a scope having a solid image sensor provided at a distal end thereof to generate image-pixel signals, an image-signal processing unit that produces a video signal based on the image-pixel signals, and a monitor for reproducing and displaying an endoscope-image in accordance with the video signal output from said image-signal processing unit, said system comprising:
a scene-changing system that changes a scene on said monitor between an endoscope-image-display scene and a patient-data-list-display scene;
a storage system that stores patient data forming a patient data list which is displayed on said monitor when the scene on said monitor is changed from said endoscope-image-display scene to said patient-data-list-display scene by said scene-changing system;
a selection system that selects individual patient data from said patient data list displayed on said monitor; and
a display-control system that displays said selected individual patient data together with the endoscope-image on said monitor when the scene on said monitor is changed from said patient-data-list-display scene to said endoscope-image-display scene by said scene-changing system.

2. An electronic endoscope system as set forth in claim 1, further comprising an editing system that edits the patient data, forming the patient data list, stored in said storage system.

3. An electronic endoscope system as set forth in claim 1, wherein the production of the video signal is performed by said image-signal processing unit such that as much patient information as possible is included in said patient data list to be displayed on the TV monitor when the scene on said monitor is changed from said endoscope-image-display scene to said patient-data-list-display scene by said scene-changing system.

4. An electronic endoscope system as set forth in claim 1, further comprising:
a clock-pulse generator that produces first and second series of clock pulses, having different frequencies, such that the video signal is output from said image-signal processing unit to said monitor in accordance with either of said series of clock pulses, said first series of clock pulses having a higher frequency than that of said second series of clock pulses;
a clock-pulse-selection system that selects either said first or second series of clock pulses to be output from said clock-pulse generator in accordance with a number of image-pixel signals obtained from said image sensor; and
a clock-pulse-selection-controller that controls said clock-pulse-selection system such that said first series of clock pulses having the higher frequency is forcibly output from said clock-pulse generator whenever the scene on said monitor is changed from said endoscope-image-display scene to said patient-data-list-display scene by said scene-changing system.

5. An electronic endoscope system as set forth in claim 1, wherein said selection system includes:
an indicator system that visually indicates a patient data to be selected from said patient data list;
a manual operation system that controls the indication of the patient data to be selected from said patient data list; and
a manual settlement system that manually settles the indication of the patient data to be selected from said patient data list.

6. An electronic endoscope system as set forth in claim 5, wherein said selection system further includes:
an editing system that edits the patient data, forming the patient list patient, stored in said storage system; and
a determination system that determines whether editing of said patient data is performed by said editing system after an activation of said manual settlement system, the editing of said patient data being settled by an activation of said manual settlement system when the performance of the editing of said patient data is confirmed by said determination system.
